(19) United States
(12) Patent Application Publication
(10) Pub. No.: US 2009/0055963 A1
(43) Pub. Date: Feb. 26, 2009

(54) Systemic gene silencing in plants

(75) Inventors: Christophe Lacomme, Invergowrie (GB); Mark Andrew Taylor, Dundee (GB); Paul Birch, Dundee (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: SCOTTISH CROP RESEARCH INSTITUTE, Invergowrie, Dundee (GB)

(21) Appl. No.: 11/578,497

(22) PCT Filed: Apr. 12, 2005
(86) PCT No.: PCT/GB2005/001388

§ 371 Date: Jan. 25, 2007

Related U.S. Application Data
(60) Provisional application No. 60/561,690, filed on Apr. 12, 2004.

(30) Foreign Application Priority Data
Apr. 13, 2004 (GB) 0408109.7

(51) Int. Cl. C12N 15/82; C12N 15/00; C12N 5/04; A01H 5/00

(52) U.S. Cl. 800/278; 435/320.1; 435/419; 800/298

(57) ABSTRACT

A novel VIGS vector is described based on pGR106. The vector includes a nucleotide silencing sequence which is homologous or complementary to a target gene of a host cell. The vector can induce gene silencing in whole plants and microplants and is also effective in the tubers of Solanum spp.

The present invention relates to the use of a viral vector in inducing gene silencing in plants.

“Gene silencing” occurs when a nucleic acid sequence bearing sufficient homology hybridises to an RNA transcribed from a gene. The duplex formed, recognised as an aberrant RNA structure, will subsequently trigger the degradation of the homologous RNA transcript. As a consequence of this post-transcriptional homology-dependant mRNA degradation, the corresponding gene is said to be “silenced” as its expression is substantially reduced. Gene silencing is recognised to have great potential utility in ascribing function to identified genes. However, there remains considerable difficulty in reliably producing gene silencing against a target gene.

Virus induced gene silencing (VIGS) is increasingly being used to generate transient loss-of-function assays to assess gene function, as a more rapid alternative to stable transformation (Baulcombe, 1999; Lu et al., 2003a). VIGS triggers an RNA-mediated defence mechanism directly targeting the integrity of the invading viral genome. This sequence-specific phenomenon lowers the titre of the invading virus through an endogenous RNAse-inducible mechanism leading to viral RNA degradation (Baulcombe, 1999; Goldbach et al., 2003). By introducing host cDNA fragments within the viral genome, it is possible to redirect this mechanism to corresponding endogenous host mRNAs, therefore providing a means to down-regulate host gene expression.

VIGS vectors such as potato virus X (PVX) or tobacco rattle virus (TRV) have been optimised in the permissive host Nicotiana benthamiana (Ratcliff et al., 2001). Recent examples show that VIGS can be extended to crop species such as tomato using a previously characterized TRV VIGS vector (Liu et al., 2002), or developed for monocot species such as barley, using barley stripe mosaic virus (BSMV, Holzberg et al., 2002).

The effectiveness of a VIGS vector relies firstly on the ability of the virus to replicate and accumulate to sufficient levels in the host plant to generate dsRNA molecules that initiate silencing, as recent studies have shown that dsRNA generation is a limiting factor in VIGS (Lacomme et al., 2003). Secondly, the virus must be devoid of strong genome-based, post-transcriptional gene silencing (PTGS)-suppressors such as HC-Pro (potyviruses) or 2b (cucumoviruses) (Voinnet et al., 1999) that protect the virus against this RNA-mediated resistance mechanism. Given these conditions, it is likely that a plant virus that is able to trigger a significant VIGS response in the host N. benthamiana, has the potential to be used for VIGS in a distinct susceptible host.

As some crop species develop unique organs like tubers or edible fruits, model plants such as Arabidopsis or Nicotiana are often of limited relevance. Potato, which is a member of the Solanaceous family, is the world's fourth largest crop. Furthermore, global production is increasing by 4.5% annually (http://www.cipotato.org/potato/POTstats.htm). The availability of a VIGS vector for potato would significantly accelerate the discovery of genes integral to tuber quality or resistance to potato-specific pathogens, for example.

WO 98/36083 refers to inducing gene silencing in plants. However, the approach described relies upon the creation of a transgenic plant having the sequence encoding the interfering nucleic acid to be stably integrated into the genome of the plant.

WO 99/15682 describes a transient method of gene silencing in plants.

We have now found that a PVX-based VIGS vector is effective in triggering gene silencing in diploid (wild type) and tetraploid (cultivated) Solanum sp. Unexpectedly, the gene silencing observed extends not only to the foliar tissues but also to tubers. In addition, such characteristics were observed on in vitro propagated plants. Both leaves from in vitro propagated plants (from several generations of plant micropropagation) and in vitro generated microtubers were efficiently silenced.

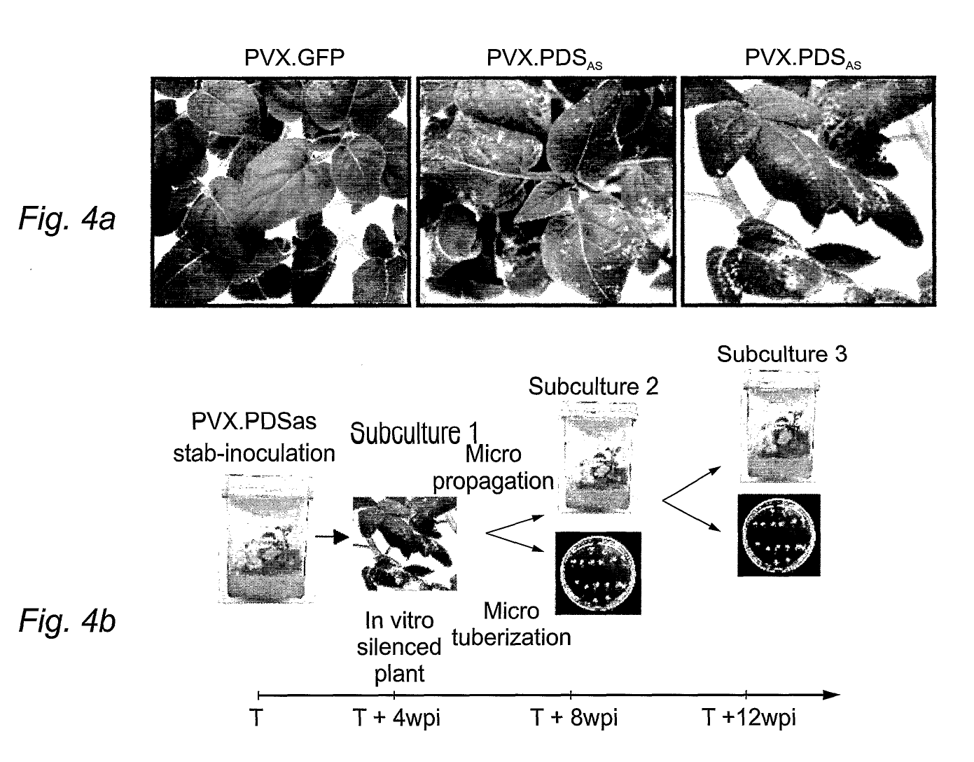

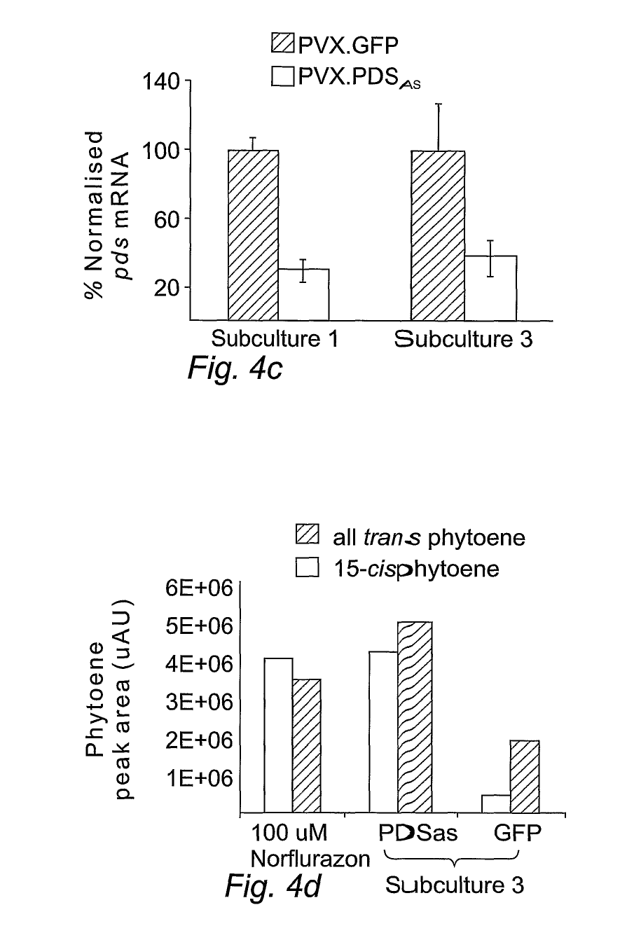

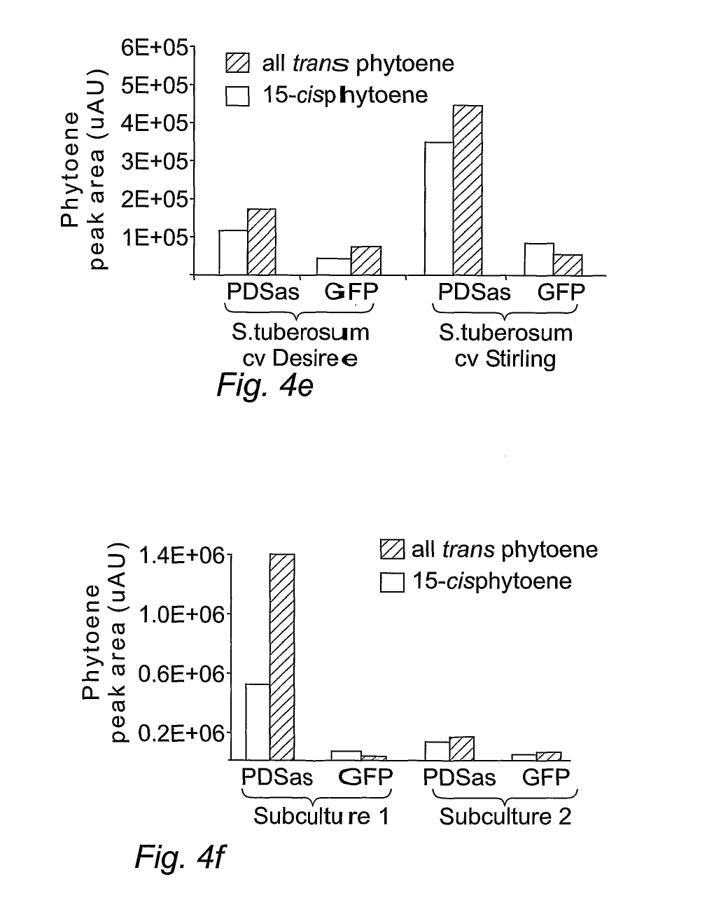

Much research in potato is directed at investigating tuber-associated traits such as tuber life-cycle, improving storage organ quality and resistance to phytopathogens. However, a major drawback is the variability in tuberization time, and the glasshouse space required in making such reverse genetics approach. In vitro material provides a means to substantially reduce glasshouse space requirements for high throughput functional studies. Moreover, in vitro grown potato offers an interesting alternative, as in vitro microtuberization is synchronized and controlled (FIG. 4b; Xu et al., 1998) allowing rapid direct screening for phenotypical modification of microtubers development. Indeed, fully developed microtubers were obtained by 9 weeks of culture (FIG. 4b), whereas, in glasshouse conditions, fully developed tubers were obtained by 12 to 15 weeks post-sowing.

In summary, the microtuberization system, in conjunction with VIGS, has a number of potential benefits compared with analysis of tubers produced conventionally in glasshouse conditions. Microtubers develop rapidly in a relatively synchronous manner under controlled tissue-culture conditions. This enables easier identification of tuber phenotype alterations, making it more amenable for characterization of gene function associated to the previously mentioned traits and high-throughput approaches such as whole-transcriptome analysis of the effect of down-regulation of one or several gene expression.

In one aspect, the present invention provides a recombinant vector, said vector comprising the vector pGR106 containing a polynucleotide silencing sequence complementary or homologous to at least part of a target gene. pGR106 has been previously described (Lu et al, 2003b); and is a derivative of a previously described PVX vector (see Chapman et al, 1992; GenBank accession number AY297843). As explained above, this vector is able to induce gene silencing in tubers as well as foliar tissues and is effective in both whole plants and microplants.

The silencing sequence may be present in a sense or anti-sense orientation, and when expressed causes silencing of the target gene in a host cell.

Generally expression of the silencing sequence is under the control of a promoter, and optionally an enhancer. The promoter may be constitutive (such as the viral CaMV35S promoter; Odell et al, 1985) or be regulatable or inducible. Transcription under the control of an inducible promoter is “switched on” or increased in response to an applied stimulus. The nature of the stimulus varies between promoters. Suitable promoters are know in the art and include promoters such as the DEX promoter (Aoyama and Chua, 1997) or the ethanol-inducible a1cA-a1cR system (Roslan et al., 2001).

In one embodiment the silencing sequence is homologous or complementary to a part of the target gene which corresponds to at least 50% of the full target gene mRNA transcript sequence, preferably at least 60%, 70% or 80% of the full target gene mRNA transcript sequence. More preferably the silencing sequence is homologous to or complementary to 90% or more of the target gene mRNA transcript sequence, for example 95% or 98% of the target gene mRNA transcript sequence.

It is not necessary for 100% homology or complementary correspondence of the silencing sequence to the target gene mRNA transcript, but the degree of homology/complementary correspondence should be sufficient to ensure that the silencing sequence binds to the mRNA transcript of the target gene sufficiently tightly to form a dsRNA molecule and initiate silencing.

It is generally preferable that the silencing sequence bears at least 80% homology to the part of the target gene sequence selected, for example exhibits 85%, 90% or 95% homology thereto. Optionally, the silencing sequence includes the ATG start codon of the target sequence.

The target gene may be any gene of interest or where reduction or elimination of the levels of the transcribed polypeptide thereof causes a desired effect. Examples include both endogenous plant genes (such as genes involved in carotenoid biosynthesis: for example phytoene synthase, phytoene desaturase, Bush et al, 2002; starch biosynthesis: for example granule-bound starch synthase, Kuipers et al, 1994; or ADP-glucose pyrophosphorylase, Muller-Rober et al, 1992) as well as transgenes present in transgenic plants. The target gene may be an enzyme, a structural protein or the like, and may be associated with one or more of the following processes: general metabolism (such as sugar, starch, carotenoid, etc.), ripening, pollen or seed formation, general plant development, specific organ development (such as tubers), male sterility or disease resistance. The system described here could also be used to control other traits.

The target gene will usually be expressed in a plant host cell, usually a whole plant or a microplant. In one embodiment the plant host cell is a cell of Solanum spp.

Suitable target genes include the zeaxanthin epoxidase (zep) gene, involved in carotenoid biosynthetic pathway, and the granule-bound starch sythase (GBSS) gene, involved in amylose biosythesis. Where the zeaxanthin epoxidase gene is the target gene, a suitable silencing sequence is SEQ ID No. 13. Where the granule-bound starch synthase gene is the target gene, a suitable silencing sequence is SEQ ID No. 14.

In another aspect, the present invention provides the use of pGR106 as a VIGS vector. pGR106 has found particular utility as a VIGS vector in Solanum spp. host cells, typically plants or microplants.

In one aspect, the present invention therefore provides a method of silencing a target gene expressed by Solanum spp., said method comprising operably linking a silencing sequence which is complementary or homologous to at least part of said target gene into a vector, wherein said vector is derived from pGR106. In one embodiment, the target gene is on endogenous gene which is present in the genome of Solanum spp, (for example the zep gene or GBSS gene).

The term “operably linked” means that the polynucleotide silencing sequence can be expressed from the vector. Of course, expression may be made dependent upon the presence of an applied stimulus depending upon the nature of the promoter used and the presence of any enhancer or other control element.

The method is suitable for use with whole plants, but may also be used in microplants. Microplants have the advantage of more rapid development and allow easier phenotype characterisation.

The method may be used to effect gene silencing in tuber (or microtuber) cells.

The method may be used to effect gene silencing in foliar cells.

In a further aspect, the present invention provides a method of producing a VIGS vector able to induce gene silencing in a plant host cell, said method comprising operably linking a polynucleotide silencing sequence to a promoter in the vector pGR106.

The silencing sequence is homologous to a part of the target gene which corresponds to at least 50% of the full target gene mRNA transcript sequence, preferably at least 60%, 70% or 80% of the full target gene mRNA transcript sequence. More preferably the silencing sequence is homologous to or complementary to 90% or more of the target gene mRNA transcript sequence, for example 95% or 98% of the target gene mRNA transcript sequence.

The degree of homology or correspondence of the silencing sequence to the target gene or its mRNA transcript need not be 100%, but should be sufficient to produce a dsRNA and initiate gene silencing.

Optionally the target gene selected is a gene expressed by Solanum spp, and may be expressed in foliar tissue and/or tuber cells.

In a further aspect the present invention provides a plant cell transfected with a recombinant vector as described above.

In one embodiment the plant cell forms part of a whole plant or a microplant, and may form part of the foliar tissues or tuber/microtuber.

Optionally the plant cell is a cell of Solanum spp.

Viewed from a further aspect, the present invention provides a host plant cell. The host plant cell will often be in the form of a whole plant or a microplant and may be, for example, of Solanum spp.

The present invention will now be further described with reference to the following, non-limiting, examples in which:

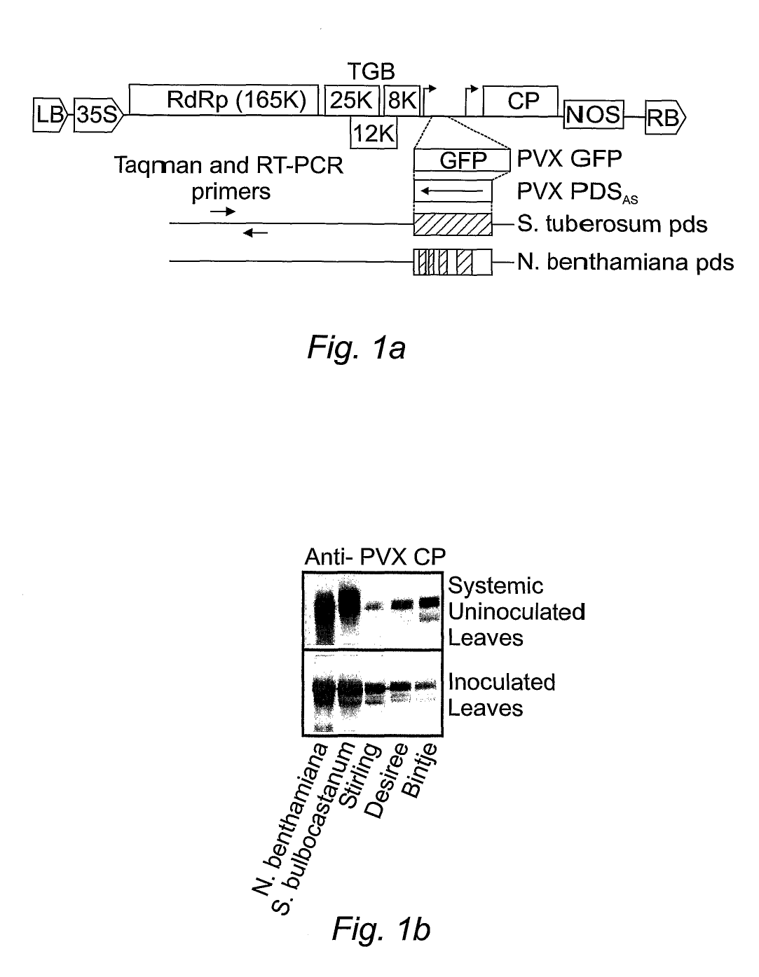

FIG. 1 represents a PVX VIGS vector accumulation in a range of Solanum species.

(a) Schematic representation of a PVX vector in the pGREEN0000 binary plasmid: 35S=35S promoter of cauliflower mosaic virus; RdRp (165K)=PVX 165K RNA-dependent RNA polymerase; TGB 25K, 8K, 12K=PVX triple gene block movement proteins; CP=viral coat protein gene; NOS=nopaline synthase transcriptional terminator; LB and RB=left and right T-DNA border sequences. Schematic representation of Solanum tuberosum and Nicotiana benthamiana full-length pds cDNAs (black line). The pds cDNA region from S. tuberosum cloned into PVX is represented as a dark grey box. The corresponding region from N. benthamiana is boxed in light grey, sequence stretches of more than 21 nucleotides identical to S. tuberosum are represented as dark grey rectangles. Arrows indicate the position of the primers used for RT-PCR and real time RT-PCR.
(b) Western blot analysis of PVX CP protein accumulation in N. benthamiana, S. bulbocastanum and S. tuberosum cvs in inoculated and systemic upper uninoculated leaves at 15 dpi.

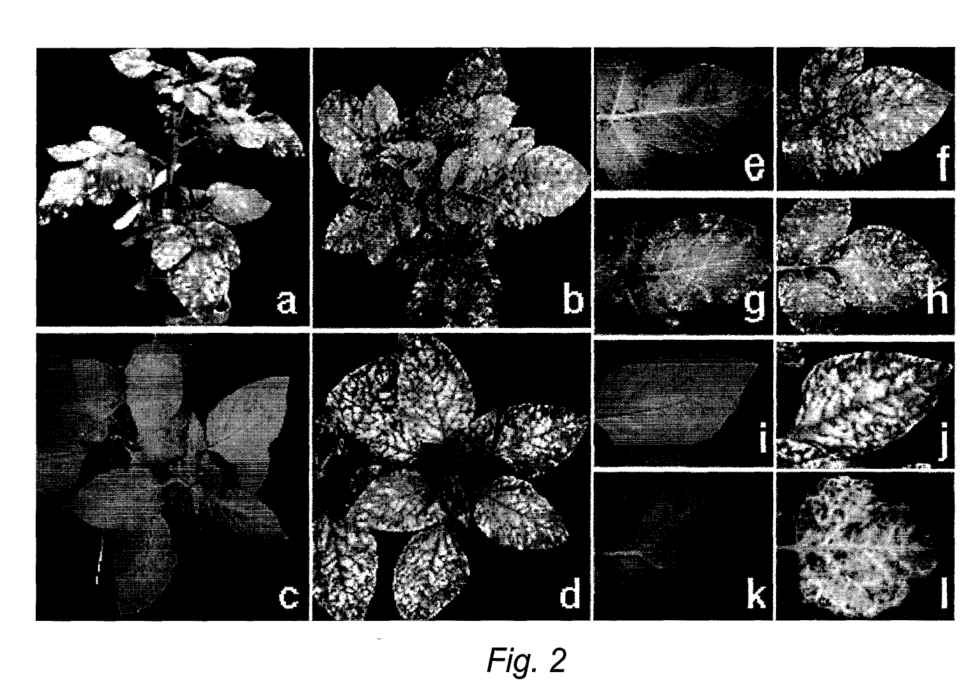

FIG. 2 shows that PVX.PDS_{AS} triggers VIGS in diploid and tetraploid Solanum species. Photobleaching phenotypes observed by 21 dpi on tetraploid S. tuberosum cv Bintje (a, b) and diploid S. bulbocastanum (d) and PVX.GFP control infected plant (c). Close-up on photobleached leaves of S. tuberosum cvs Bintje (f), Stirling (g), Desiree (h), S. bulbocastanum (j), N. benthamiana (l) and on symptomless PVX.GFP infected leaf of cv Bintje (e) and S. bulbocastanum (i). Uninfected N. benthamiana leaf (k).

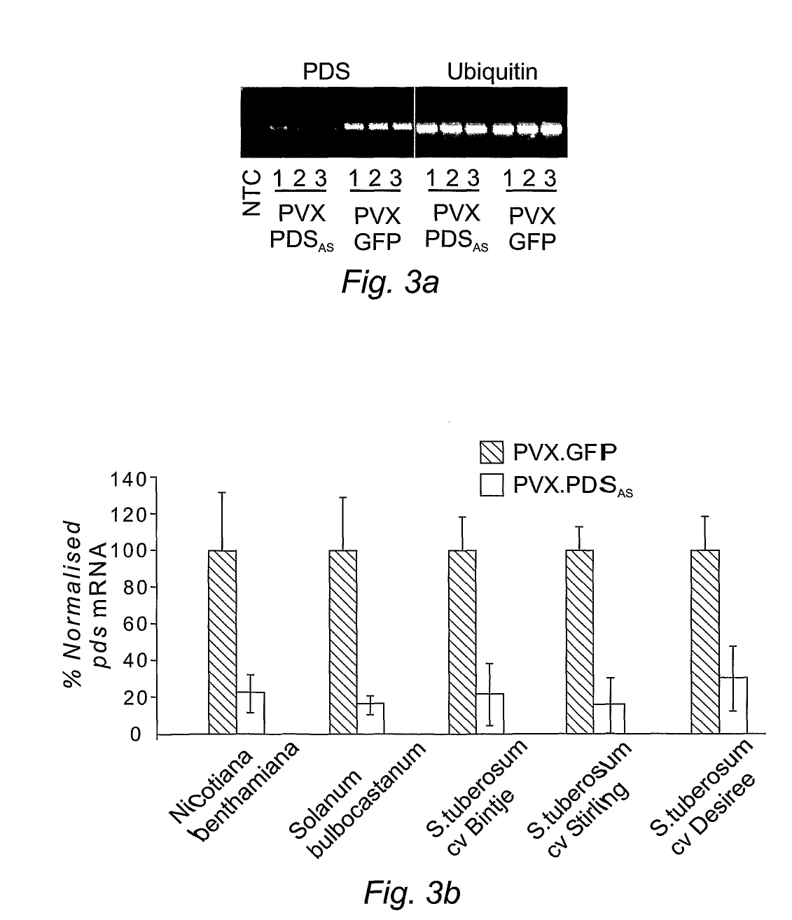

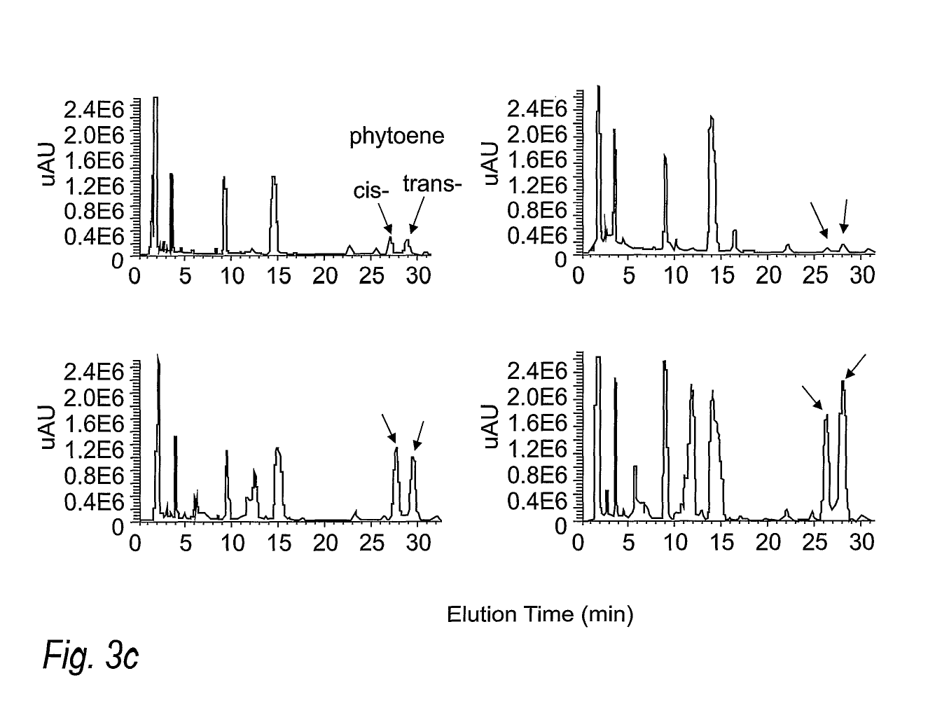

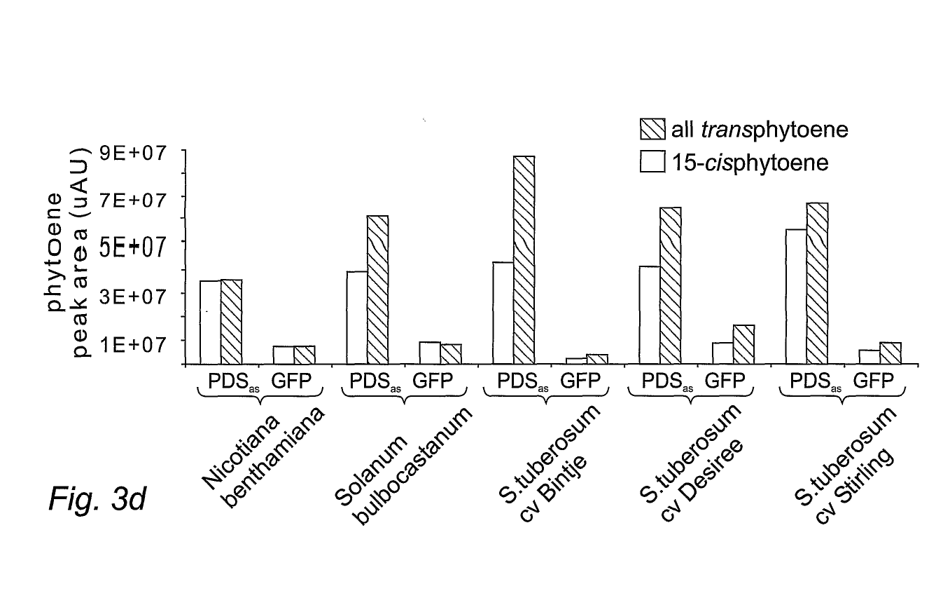

FIG. 3 represents molecular and biochemical characterization of pds VIGS in diploid and tetraploid Solanum species.

(a) RT-PCR of S. tuberosum cv Bintje pds-silenced and control plants in response to challenge with PVX constructs. Both RT-PCR products corresponding to endogenous pds and ubiquitin mRNAs have been assessed. PCR conditions ranging from 20 to 50 amplification cycles were tested in both cases. Presented here are 30 cycles corresponding to the log-linear phase of amplified PCR product in non-silenced tissues (challenged PVX.GFP construct). NTC, non-template control; replicate are leaves from 3 different challenged plants.
(b) Real-time RT-PCR determination of normalised relative amounts of pds mRNA levels in silenced and control plants challenged with PVX.PDS_{AS} or PVX.GFP constructs (21 dpi). Sampled leaves were cut in half for either RNA extraction (real-time RT-PCR) or phytoene isolation (HPLC analysis). Values are expressed in percentage of normalised pds mRNA related to PVX.GFP control. Value represent the means of at least three leaves from different plants per construct per experiment ±SE. For each sample, real-time RT-PCR was carried out in triplicate.
(c) HPLC analysis of phytoene accumulation induced by PVX.PDS_{AS} VIGS vector. Typical HPLC profile from S. tuberosum cv Bintje and N. benthamiana is presented here. As before, silenced leaves from plant challenged with PVX.PDS_{AS} and corresponding leaves from PVX.GFP plants were sampled at 21 dpi. Arrows indicate peaks for both 15 cis-phytoene and all trans-phytoene.
(d) Quantification of phytoene accumulation in silenced and control Solanum species and N. benthamiana. Values for 15—cis-phytoene and trans-phytoene are HPLC area units (Arbitrary Units) and represent the means of at least three leaves from different plants per construct per experiment (SE<20%).

FIG. 4 shows systemic pds silencing in tubers and in vitro propagated plants and microtubers.

(a) Photobleaching phenotypes observed on in vitro propagated S. tuberosum cv Desiree after 3 subcultures (representing 12 weeks of in vitro propagation post-challenge with PVX.PDSas, middle panel) or 1 subculture (4 weeks post-challenge with PVX.PDS_{AS}, right panel). PVX. GFP control infected plant is shown on the left panel.
(b) Schematic representation of in vitro culture and generation of in vitro grown microtubers. Time scale in week post-challenge with PVX construct is presented.
(c) Real-time RT-PCR determination of normalised relative amounts of pds mRNA levels in silenced and control leaves from in vitro grown S. tuberosum cv Desiree plants challenged with PVX.PDS_{AS} or PVX.GFP constructs. Leaves were harvested after 4 weeks post challenge (28 dpi, subculture 1) or after 12 weeks post challenge (more than 80 dpi, subculture 3). As before, sampled leaves were cut in half for either RNA extraction (real-time RT-PCR) or phytoene isolation (HPLC analysis). Values are expressed in percentage of normalised pds mRNA related to PVX.GFP control. Values represent the means of at least three leaves from five different in vitro plants per construct per experiment ±SE. For each sample, real-time RT-PCR was carried out in triplicate.
(d) Quantification of phytoene accumulation in leaves of in vitro S. tuberosum cv Desiree challenged with PVX.PDS_{AS} or PUX.GFP. Phytoene accumulation was monitored in norflurazon-treated in vitro plants. Values for 15 cis-phytoene and trans-phytoene are HPLC area units (Arbitrary Units) and represent the means of at least three leaves from different plants per construct per experiment (SE<20%).
(e) Quantification of phytoene accumulation in tubers (approximately 15 weeks after challenge with PVX constructs) from silenced (PVX.PDS_{AS} challenged) and control (PVX.GFP challenged) S. tuberosum cv Desiree and Stirling. Values for 15 cis-phytoene and trans-phytoene are HPLC area units (Arbitrary Units) and represent at least ten tubers from at least two different plants per construct per experiment (SE<20%).
(f) Quantification of phytoene accumulation in in vitro generated microtubers from silenced and control S. tuberosum cv Desiree, after 8 weeks (subculture 1) or 12 weeks (subculture 2) initial challenge with PVX constructs. Values for 15 cis-phytoene and trans-phytoene are HPLC area units (Arbitrary Units) and represent 15 to 20 microtubers generated from four to six different in vitro grown plants per construct per experiment (SE<20%).

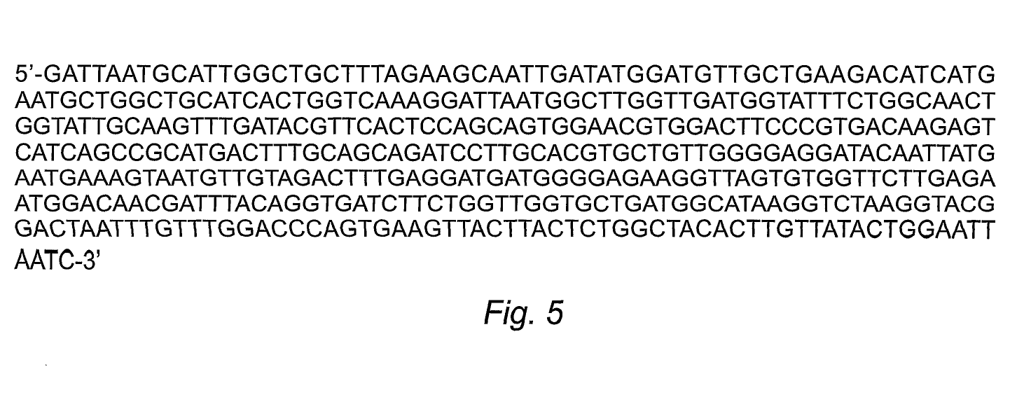

FIG. 5 shows the nucleotidic sequence of the 428 bp cDNA fragment corresponding to zeaxanthin epoxidase (zep) Solanum phureja DB375\1 cDNA clone (Morris et al., 2004, homologous to TIGR database EST498983, GenBank accession number BG591141) (SEQ ID No. 13) subcloned in antisense orientation into pgR106 to generate VIGS construct PVX.ZEPas.

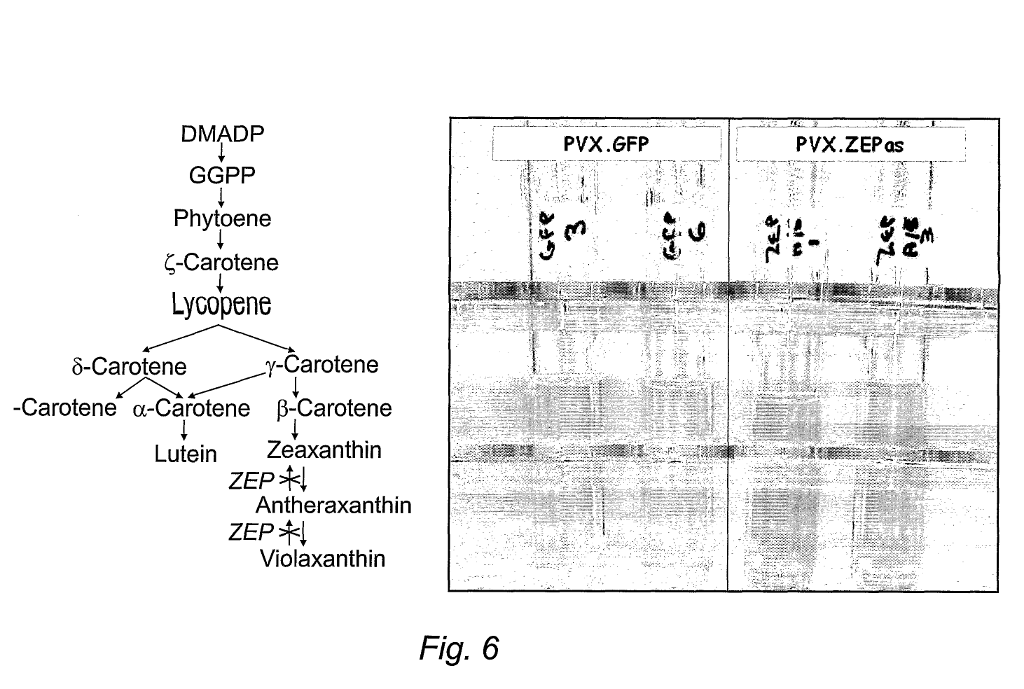

FIG. 6 shows that PVX.ZEPas construct trigger VIGS of the corresponding zeaxanthin epoxidase (zep) gene in potato tubers. Left panel: carotenoid biosynthetic pathway in potato showing the role of the ZEP enzyme. ZEP is involved in two successive steps of conversion of zeaxanthin to antheraxanthin and antheraxanthin to violaxanthin. Right panel: acetone extracts of lyophilised tubers from zep silenced (PVX.ZEPas) and control PVX infected (PVX.GFP) samples. Note the yellow coloration of the tuber acetone extracts indicating of an increase of total carotenoid content in zep silenced tubers originating from two independent PVX.ZEPas infected plants in comparison to pale yellow coloured PVX.GFP control tubers.

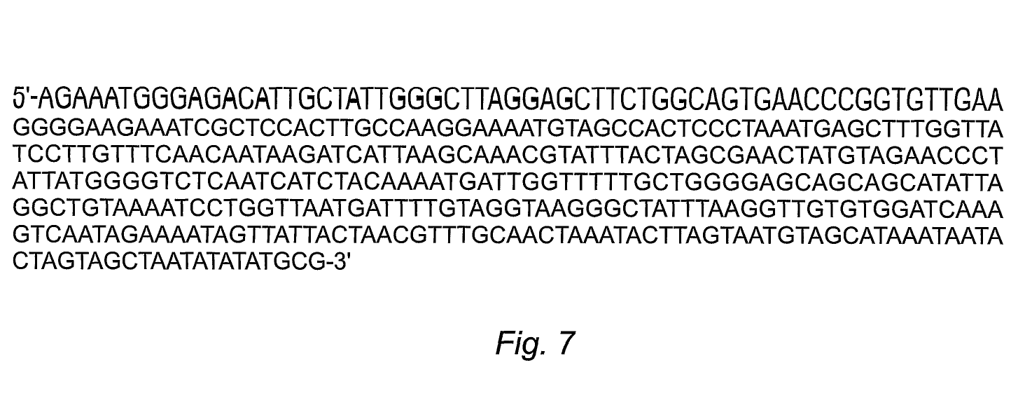

FIG. 7 shows nucleotidic sequence of the 392 bp granule-bound starch synthase (GBSS) potato cDNA fragment (GenBank accession number X58453) (SEQ ID No. 14) subcloned in antisense orientation into pgR106 vector to generate VIGS construct PVX.GBSSas

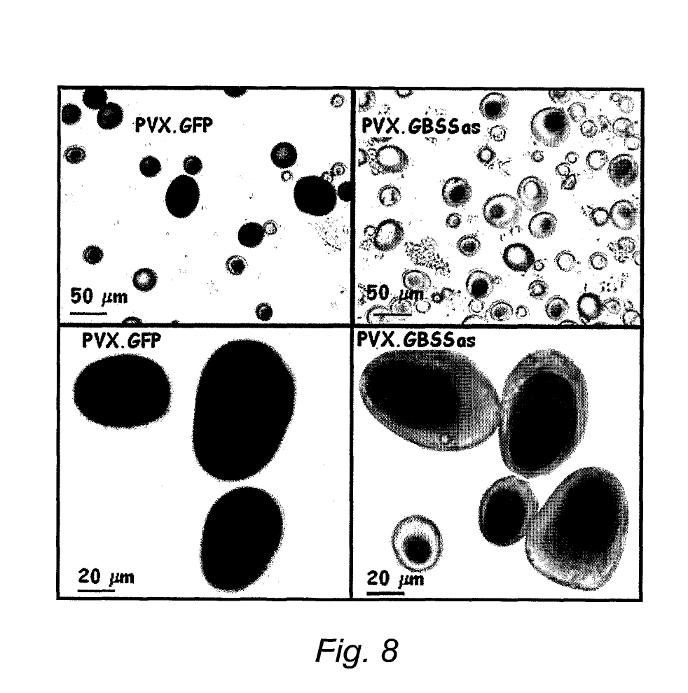

FIG. 8 shows that PVX.GBSSas construct trigger VIGS of the corresponding granule-bound starch synthase (GBSS) gene in potato tubers. Iodine staining of starch granules from control (left panel, PVX.GFP) or GBSS silenced (right panel, PVX.GBSSas) samples originating either from in vitro generated microtubers (upper panels, scale bars represent 50 μm) or mature tubers from glasshouse-grown potato plants (lower panels, scale bars represent 20 μm). Note the pale-red concentric growth rings with blue core (upper right panel) and mixed thin-blue and red concentric growth rings with blue core (lower right panel) from iodine-stained starch granules originating from PVX.GBSSas (upper and lower right panels) infected plants characteristic of reduced amylose content as a consequence of GBSS silencing in microtubers and tubers starch granules. In contrast, starch granules with unaltered amylose content from unsilenced control (PVX.GFP upper and lower left panels) tubers appear blue after Lugol staining.

### EXAMPLES

### Experimental Procedures

### Construction of PVX-Derived Vectors

The PVX vector (pGR106, Jones et al., 1999; Lu et al., 2003b) was obtained from David Baulcombe (Sainsbury Laboratory, Norwich, UK). PVX.GFP was generated by cloning a PCR fragment amplified from a gfp cDNA template (GenBank accession number U62637, Crameri et al., 1996) using specific oligonucleotide primers incorporating AscI and NotI restrictions sites respectively at the 5′- and 3′-termini for cloning into pGR106. The construct PVX.PDS_{AS} was generated by cloning in antisense orientation into pGR106 a NotI-AscI 412 bp pds cDNA fragment, corresponding to nucleotides 1133-1529 from Solanum tuberosum pds cDNA (GenBank accession number AY484445).

### Agrobacterium Infection of Plants

Agrobacterium tumefaciens strain LB4404, carrying the helper plasmid pSoup (Hellens et al., 2000) was transformed with constructs PVX.GFP or PVX.PDS_{AS}. Agroinfiltration of N. benthamiana and Solanum species with PVX vector was performed as previously described (Lu et al., 2003a). For in vitro agroinoculation, plated individual agrobacteria transformed with PVX.GFP or PVX.PDS_{AS} constructs were picked with a sterile tip and punched onto a leaf of a 2-week old potato plant (Takken et al., 2000).

RNA Extraction and cDNA Synthesis

Total RNA was extracted from frozen control and silenced leaves using the Qiagen RNeasy plant mini kit (Qiagen Ltd), following the manufacturer's instructions. DNAseI treatment and first strand cDNA synthesis were as previously described (Lacomme et al., 2003).

### Immunoblot Analysis

Protein extraction and western blot analysis were as previously described (Lacomme and Santa Cruz, 1999). Membranes were probed with rabbit polyclonal antiserum, raised against the PVX CP as previously described (Santa Cruz et al., 1996)

### RT-PCR and SYBR Real-Time RT-PCR Experiments

For RT-PCR analysis, primers that anneal outside the region of the pds cDNA cloned into the virus vectors to trigger silencing (nucleotides 1126-1516) were used to ensure that only the endogenous pds mRNA is reverse-transcribed as indicated in FIG. 1a. Potato ubiquitin cDNA (GenBank accession number BQ045862) was used as an internal constitutively expressed control. First-strand cDNA was used as a template for PCR amplification through 20, 25, 30, 40 and 50 cycles. As 30 cycles of amplification was within the log-linear phase of pds PCR product amplification in the non-silenced control samples (data not shown), these conditions were selected for comparison of relative accumulation of both pds and ubiquitin mRNAs in all samples. The following primers were used: RTPoPDSfor (5′-CTC GAG GTC GTC TTC TTT GG-3′ SEQ ID No. 1); RTPoPDSrev (5′-GTT TAG TTG GGC GTG GAG AA-3′ SEQ ID No. 2); RTPoUBIfor (5′-GCA GTT GGA GGA CGG AC-3′ SEQ ID No. 3); RTPoUBIrev (5′-GGC CAT CTT CCA ACT GTT TC-3′ SEQ ID No. 4). Similarly, for SYBR real-time RT-PCR experiments, primer pairs were designed outside the region of the S. tuberosum pds cDNA targeted for silencing (FIG. 1a) and for the internal control ubiquitin cDNA using the Primer Express software supplied with the ABI PRISM 7700 Sequence Detection System (Applied Biosystems, USA) following the manufacturer's guideline for primer design. The following primers were used: StPDSfwd (5′-CCA AGA CCA GAG CTA GAC AAT ACA GT-3′ SEQ ID No. 5); StPDSrev (5′-CCA CCC AAA CCT GCA-3′ SEQ ID No. 6); StUBIfwd (5′-ACA CCA TTG ATA ATG TCA AGG CTA AG-3′ SEQ ID No. 7); and StUBIrev (5′-GCC ATC CTC CAA TTG CTT TC-3′ SEQ ID No. 8). The GenBank accession numbers for N. benthamiana pds and ubiquitin cDNA are as previously mentioned (Lacomme et al., 2003). Primer concentrations giving the lowest threshold cycle (Cₜ) value were selected for further analysis. Detection of real-time RT-PCR products, calculations and statistical analysis were performed as previously described (Lacomme et al., 2003).

### Plant Material and Growth Conditions

All work involving virus-infected material was carried out in containment glasshouses. Potato cultivars Stirling, Bintje and Desiree were micropropagated in sterile conditions by removing 5 cm of young potato stems from virus-tested potato plants from the Scottish Agricultural Science Agency (SASA, Edinburgh, Scotland), removing leaves and dividing the stem into individual pieces each containing a node with an axillary bud. Six stem pieces were cultivated per Petri dish containing 20 mL of MS medium (Murashige and Skoog, 1962) supplemented with 20% sucrose, 0.8% bacto agar (Difco) and adjusted to pH 5.8, sealed with Nescofilm (Bando Chemical Ind., Japan). Plants were then grown at 22° C. with 16 h photoperiod and 110 μE m⁻² s⁻¹. When potato plants were 3 to 4 weeks old, they were then transferred to soil in controlled environment chambers with a 16 h photoperiod (22° C., light intensity ranging from 400 to 1000 μE m⁻² s⁻¹).

### In Vitro Microtuberization

After 28 days culture, potato plants were divided into single nodes and placed on Murashige and Skoog medium supplemented with 60% sucrose, 7 mM chlorocholine chloride (CCC), 8 μM benzylaminopurine (BAP), 0.8% agar (Difco). CCC and BAP were added aseptically after autoclaving by filter sterilising. Fifteen nodes were placed in each dish and the dishes were sealed with Nescofilm. The cultures were then placed in an incubator in an 8 h photoperiod at 80 μm⁻² s⁻¹ at 16° C. for 7 days prior to a total darkness at the same temperature for a further 28 days. The microtubers were then harvested and frozen for further analyses.

Extraction and HPLC Analysis of Phytoene from Transfected Plants

Phytoene was extracted from infected leaves, tubers and microtubers. The method used was as described in Holzberg et al. (2002). 50 mg of freeze-dried leaves (3 leaves from different plants), 100 mg of freeze-dried tubers (representing at least 10 mature tubers), or 100 mg of freeze-dried in vitro generated microtubers (representing 15 to 20 microtubers deriving from at least four independent in vitro grown plants) were extracted in 100% methanol. The samples were then centrifuged for 5 min at 4,000 rpm at 4° C. The supernatants were dried under a stream of nitrogen. Residues were redissolved in 500 μL of 100% methanol and 10 μL were separated by HPLC (Thermo Finnigan Surveyor system, USA) with a Phenomenex 2×250 mm C-18 column using acetonitrile/methanol/2-propanol (85:10:5, v/v) at a flow rate of 300 μl.min⁻¹. Phytoene was detected with a photodiode array detector using UV absorption at 285 nm and identified by comparing peak retention times with norflurazon-treated and untreated potato plants (Fraser et al., 2000; Holzberg et al., 2002).

### Results and Discussion

### PVX Infects Both Diploid and Tetraploid Solanum Species

A susceptible host is a prerequisite for the development of an efficient VIGS system, as viral replication and in planta accumulation of the virus, condition the generation of dsRNA molecules that initiate silencing (Voinnet, 2001). Some plant viruses such as PVX have a relatively broad host-range, including several Solanaceous species (Brunt et al., 1996). In this study, a previously described binary PVX-expression vector (Jones et al., 1999; Lu et al., 2003b) was tested for its capacity to infect both wild diploid and cultivated tetraploid Solanum species. Cultivars were selected either due to their ability to be stably transformed and propagated in vitro (Solanum tuberosum L. cv Desiree), or the differential interactions occurring between either susceptible or resistant cultivars (S. tuberosum L. cvs Bintje and Stirling, respectively; Birch et al., 1999) to Phytophthora infestans, or as a potential source of novel resistance genes to P. infestans (Solanum bulbocastanum, Song et al., 2003).

Infectivity of PVX in these Solanum hosts was investigated using a PVX construct carrying a GFP insert (PVX.GFP construct, FIG. 1a). Following agroinoculation of young source leaves of potato plantlets, both infiltrated and systemic upper-uninoculated leaves were harvested. Virus accumulation was monitored by immunodetection of viral PVX coat protein (CP) by western blotting (FIG. 1b).

On the basis of semi-quantitative western analysis, more PVX CP was detected in both inoculated and upper-uninoculated (systemic) leaves in S. bulbocastanum than S. tuberosum L. cvs Desiree, Stirling or Bintje (FIG. 1b lower and upper panel). In S. bulbocastanum PVX accumulation was comparable to that observed in N. benthamiana at the same time post-inoculation on both inoculated and systemic leaves (FIG. 1b). In all Solanum species and cultivars that were tested, PVX-CP was also detected in systemic leaves by 14 dpi (FIG. 1b upper panel). Therefore, all plants tested tolerate substantial PVX accumulation.

### The PVX Vector Triggers VIGS of Endogenous pds in Foliar Tissues in Solanum Species

The silencing effectiveness of the binary PVX vector was assessed by its ability to silence an endogenous pds gene in these different Solanum species. Down-regulation of endogenous pds gene expression leads to a characteristic photobleaching phenotype, therefore providing an indication of gene silencing (Kumagai et al., 1995; Ratcliff et al., 2001). As RNA silencing is homology-dependant, a potato pds cDNA fragment was subcloned into PVX. The cDNA fragment selected was a region showing sequence identity of 91% with an N. benthamiana pds cDNA (including stretches of 24, 26, 33 and 47 nucleotides of 100% identity between both cDNAs, FIG. 1c). This would allow silencing of the corresponding genes in both species to compare the relative VIGS. The cDNA region was subcloned in antisense orientation into the PVX vector (construct PVX.PDS_{AS}, FIG. 1a). Following challenge with PVX.PDS_{AS}, photobleaching was observed on all N. benthamiana plants by 12 to 15 days post-inoculation, suggestive of pds silencing (FIG. 21). When the Solanum species and cultivars were infected with PVX.PDS_{AS}, white patches of photobleached tissues were observed by 3 weeks post-inoculation in all infected plants (S. bulbocastanum, FIGS. 2d and 2j; S. tuberosum L. cvs Bintje, FIG. 2a, 2b, 2f; Stirling, FIG. 2g; and Desiree, FIG. 2h) as opposed to plants infected with PVX.GFP where no symptoms of PVX infection were visible (S. bulbocastanum, FIG. 2c and S. tuberosum L. cv Bintje, FIG. 2e). In silenced plants, the degree of photobleaching varied, however, from covering most of the leaf surface for S. bulbocastanum (FIGS. 2d and 2j), to patches uniformly distributed on the leaf surface close to leaf veins for S. tuberosum cvs Bintje, Stirling and Desiree (respectively FIGS. 2f, 2g and 2h). This systemic photobleaching was sustained for the duration of the experiment (up to 3 months post-challenge with PVX.PDS_{AS}) as silenced leaves remained photobleached and newly developing leaves underwent photobleaching as observed in the earlier stages of the VIGS response (FIG. 2a, 2b and data not shown).

VIGS effectiveness was analysed at the transcript level by monitoring pds mRNA accumulation by RT-PCR and real-time RT-PCR. Leaf samples were taken from three to six different plants challenged by either PVX.PDS_{AS} or PVX.GFP (the latter as a control of PVX infection). RT-PCR experiments detected a lower amount of pds PCR product in the silenced leaves than in the control samples (FIG. 3a). The levels of control ubiquitin RT-PCR product were similar in all samples tested (FIG. 3a). Real-time RT-PCR was then used to quantify the levels of normalised pds mRNA in both silenced and control leaf tissues. A decrease in normalised pds mRNA levels ranging from 70±17% to 84±5% was detected in silenced tissues of S. tuberosum cv L. Desiree and S. bulbocastanum (FIG. 3b) when compared with PVX.GFP-infected control leaves. A comparable decrease in normalised pds mRNA was detected between leaves of diploid S. bulbocastanum and tetraploid S. tuberosum L. cv Bintje, Stirling and Desiree (respectively 84±5%, 78±17%, 84±15% and 70±17%). This decrease in pds mRNA was similar to that observed during PVX.PDS_{AS} VIGS in N. benthamiana (78±10%, FIG. 3b), although the extent of photobleaching appeared greater than that observed with Solanum leaves (FIG. 21). This corroborates the fact that in these experiments and as previously reported (Ratcliff et al., 2001; Lacomme et al., 2003), the extent of photobleaching does not always correlate with differences in pds mRNA levels.

To further characterise the bleached phenotype in potato following pds silencing, the levels of phytoene were quantified by reverse-phase HPLC in extracts from both N. benthamiana and potato leaves challenged with either PVX.GFP or PVX.PDS_{AS} VIGS constructs. An increase in the level of phytoene, the substrate for PDS, is known to occur during VIGS of the pds gene (Kumagai et al., 1995). Phytoene also accumulates to high levels in leaves treated with the herbicide norflurazon, a chemical inhibitor of PDS activity (Kumagai et al., 1995). The peaks in the HPLC chromatogram corresponding to cis- and trans-phytoene were identified by comparison between norflurazon-treated and untreated plants and the characteristic absorption spectra of those peaks (Fraser et al., 2000 and data not shown). Increase in phytoene accumulation was quantified as the number of area units under phytoene peaks on the HPLC chromatogram (Holzberg et al., 2002).

Typical HPLC chromatograms are presented in FIG. 3c. In both silenced PVX.PDSas infected N. benthamiana and S. tuberosum plants an increase in 15 cis- and trans-phytoene levels was observed (FIG. 3c lower left and right panels respectively) in comparison to PVX.GFP Control Plants. (FIG. 3c, upper panel). In silenced N. benthamiana leaves phytoene levels increased by 5- to 10-fold in comparison to PVX.GFP control plants (FIG. 3d). A comparable result was obtained for all Solanum species and cultivars tested (FIG. 3d). Although N. benthamiana displayed the strongest photobleaching, the increase in phytoene accumulation was lower than that observed in Solanum species and cultivars. Moreover, although S. bulbocastanum displayed the strongest photobleaching phenotype amongst the Solanum genotypes, similar levels of phytoene accumulation were observed in S. tuberosum L cv. Desiree and Stirling, and these were lower than that observed in cv. Bintje (FIG. 3d).

This indicates that the increase in phytoene level may vary from 5- to 10-fold in Solanum silenced leaf tissue despite comparable decreases in pds mRNA levels. We thus propose that a similar, if not stronger, VIGS of pds in Solanum species and cultivars triggers a milder photobleaching phenotype than observed in N. benthamiana and this indicates that leaf photobleaching is not quantitatively coupled to the degree of pds silencing between different species.

### Systemic VIGS of pds in Potato Tubers and In Vitro Generated Microtubers

We evaluated the potential of a VIGS-based approach for in vitro grown potato species by down-regulation of pds in S. tuberosum L cv Desiree micropropagated plants. In vitro material provides a means to substantially reduce glasshouse space requirements for high throughput functional studies. Stab-agroinoculation (Takken et al., 2000; Lu et al. 2003a) of leaves of in vitro grown plants with plated Agrobacteria transformed with either PVX.GFP or PVX.PDS_{AS} constructs was an effective method to generate reproducible PVX infections. By 4 weeks post-inoculation, development of systemic photobleached areas on leaves was clearly visible on PVX.PDSas inoculated plants (FIG. 4a middle and right panel) in comparison with PVX.GFP controls (FIG. 4a, left panel). Micropropagation of nodal cuttings from silenced plants led to regenerated potato plants displaying a comparable photobleaching phenotype within 3-4 weeks (FIG. 4b and data not shown). To determine whether this photobleached phenotype was maintained through several cycles of subculture, micropropagation of the original PVX.PDS_{AS} challenged plant was repeated, and a sustained photobleaching was still observed even after the fifth consecutive subculture (data not shown). The three first subcultures of in vitro grown plants challenged by either PVX.GFP or PVX.PDS_{AS} (subculture 1 and subculture 3) were analysed using real time RT-PCR. A significant decrease in pds mRNA was observed in both subculture 1 and subculture 3 (FIG. 4c; respectively 70±7% and 63±11%), the latter representing 12 weeks of in vitro propagation. Phytoene accumulation was measured in these in vitro silenced and control plants. Norflurazon treated in vitro plants developed comparable photobleaching to that observed with glasshouse grown plants, and the chromatogram peak for phytoene was identified as before (data not shown). A similar increase in 15 cis- and trans-phytoene was observed in both norflurazon-treated and PVX.PDS_{AS} silenced plants (FIG. 4d) ranging from 3-fold (trans-phytoene) to 8-fold (15 cis-phytoene) in comparison with control plants challenged with PVX.GFP (FIG. 4d) after 3 cycles of micropropagation. This indicates that pds VIGS characteristics are similar both in glasshouse and in vitro conditions and confirmed the relative stability of the VIGS phenotype observed with in vitro grown plants.

Although VIGS proved effective in potato leaves, much research in potato is directed at investigating the tuber life-cycle, improving storage organ quality and resistance to phytopathogens. Therefore it was important to determine whether gene silencing was observed in tubers. However, a major drawback is the variability in tuberization time, and the glasshouse space required in making such reverse genetics approach. In vitro grown potato offers an interesting alternative, as in vitro microtuberization is synchronized and controlled (FIG. 4b; Xu et al., 1998). Indeed, fully developed microtubers were obtained by 9 weeks of culture (FIG. 4b), whereas, in glasshouse conditions, fully developed tubers were obtained by 12 to 15 weeks post-sowing.

The systemic nature of the VIGS phenotype in tuber tissues from both glasshouse-grown plants and in vitro generated microtubers was investigated. Fully developed tubers from glasshouse plants were obtained by 3 months post-challenge with either PVX.GFP or PVX.PDS_{AS}. Similarly, mature in vitro generated microtubers derived from in vitro control and silenced plants challenged by PVX.GFP or PVX.PDS_{AS} were collected and analysed by HPLC to monitor the extent of accumulation of phytoene in these organs. HPLC phytoene profiles from tubers harvested from S. tuberosum L. cvs Desiree and Stirling plants challenged with PVX.PDS_{AS} indicated, respectively, up to a two- to five-fold increase in phytoene accumulation in comparison to control PVX.GFP infected plants (FIG. 4e). In contrast, PVX.PDS_{AS} challenged microtubers showed an accumulation of up to 20-fold more phytoene than control microtubers infected by PVX.GFP (FIG. 4f). A more marginal but significant difference (2-fold) in phytoene increase was still detected in the next generation of subcultured microtubers (subculture 2), indicating a less sustained VIGS phenotype in these tissues and experimental conditions (FIG. 4f) than observed in foliar tissues of in vitro micropropagated plants. These results indicate that systemic silencing of endogenous genes in potato, exemplified here by pds, does not only extend to foliar tissues but potentially spreads through the whole plant, including tubers. This silencing state can be transmitted and detected for several generations through vegetative propagation.

We further evaluated the robustness of the PVX VIGS-based approach for silencing in tubers and microtubers by targeting two different genes involved in two distinct biosynthetic pathways as described below.

VIGS-Based Modification of Carotenoid Content in Tubers by Silencing Zeaxanthin Epoxidase (zep) Gene.

Previous work on the genetic engineering of the carotenoid content in potato tubers on transgenic plants have reported the unexpected increase in total carotenoid as a consequence of down regulation of zeaxanthin epoxidase (zep) gene (Romer et al., 2002). This gene was selected for assessing the robustness of the PVX VIGS approach in tubers as it lead to a characteristic yellow-orange tuber flesh colour phenotype. A 428-bp zep cDNA fragment from Solanum phureja DB375\1 (SEQ ID No. 13, FIG. 5, Morris et al., 2004) was subcloned in antisense orientation into pgR106 to generate PVX.ZEPas VIGS construct.

Subcloning of the zep cDNA was achieved by excision of the 442 bp zep cDNA from the pGEM®-TEasy vector (Invitrogen) backbone by NotI digestion then ligated into a NotI digested and dephosphorylated pgR106 vector. The PVX.ZEPas construct corresponding to pgR106 vector carrying zep cDNA in antisense orientation was selected by PCR screening of recombinant E. coli XL1-Blue competent cells (Stratagene) using primer 5′-TGTACTAAAGAAATCCCCATCC-3′ (SEQ ID No. 9) complementary to PVX CP ORF and primer 5′-TATTGTATCCTCCCCAACAGCA-3′ (SEQ ID No. 10) complementary to the non-coding strand of zep cDNA.

As previously described for pds silencing, 6 potato (Solanum tuberosum cv Desiree) plantlets were agro-infiltrated with each of the PVX.ZEPas and PVX.GFP constructs. Plants were left to grow and develop tubers in glasshouse conditions as described in the Experimental procedures section. By 3 months post infection, mature tubers were harvested from each plant. Peeled whole tubers samples (pooled samples of at least three tubers from two independent plants per constructs) were freeze-dried. At this stage, powdered freeze-dried material from PVX.ZEPas samples appeared yellow to dark-yellow as opposed to white to pale-yellow for control PVX.GFP samples (data not shown).

Acetone extracts from approximately 250 mg of the powdered freeze-dried tubers samples were prepared as previously described (Morris et al., 2004). A distinctive dark yellow solution corresponding to PVX.ZEPas acetone extract contrasts with the transparent to pale-yellow colour of acetone extract from PVX.GFP control samples (FIG. 6). As previously reported (Rommer et al., 2002) this indicates of an increase of total carotenoid content in zep-silenced tubers. The result obtained from the VIGS-based down regulation of zep gene is in agreement with the previous mentioned work based on transgene-mediated down regulation of the zep gene (Rommer et al., 2002). As expected, the amount of violaxanthin was diminished dramatically and zeaxanthin levels were elevated in the co-suppressed plants. The unexpected increase in total carotenoids (up to 5.7-fold in transgenic plants) suggests that in both cases of VIGS and co-suppression of zep gene, similar effects are observed on the regulate on of the whole carotenoid biosynthetic pathway in potato tubers. Therefore VIGS mediated by PVX construct reproduce the same effects associated to the down regulation of the zep gene as observed with stable transformants, and provide a rapid way to characterize the function of genes such as those involved in carotenoid biosynthesis regulation.

### VIGS-Based Modification of Amylose Content and Quality in Tubers and Microtubers In Vitro by Silencing Granule-Bound Starch Synthase (GBSS) Gene.

To evaluate the usefulness of the PVX VIGS-based approach in modulating different biosynthetic pathways, we tested its capacity to modify starch biosynthesis. Starch, the major storage carbohydrate of higher plants, is synthesized for long-term storage in amyloplasts of potato tubers. The main components of starch are linear helical amylose and branched amylopectin, which are both glucose polymers. Reserve starch in potato tubers contains up to 25% amylose. The growth of starch granules occurs via apposition, and amylose molecules are interspersed among the radially arranged amylopectin molecules (Kuipers et al., 1994). Granule-bound starch synthase (GBSS) is involved in the biosynthesis of amylose. GBSS gene expression was shown to be inhibited after the introduction of an antisense GBSS gene in transgenic potato plants (Kuiper et al., 1994). Downregulation of GBSS results in a modification of starch content with reduced levels of amylose. As a consequence, a distinctive iodine-staining pattern of starch GBSS silenced granules that consists of a blue core at the hilum surrounded by pink-red growth rings instead of blue for the control samples, is observed (Kuipers et al., 1994). Therefore the effect of GBSS down regulation can be easily assessed by simple Lugol staining.

In order to generate PVX VIGS construct harbouring in antisense orientation a cDNA fragment of GBSS cDNA, cloning of a GBSS partial cDNA was achieved by RT-PCR amplification of the 392 bp GBSS cDNA using forward primer 5′-AAAAGCGGCCGCAGAAATGGGAGAC-3′ (SEQ ID No. 11) and reverse primer 5′-AAAAGGCGCGCCCGCATATATATTAG-3′ (SEQ ID No. 12) incorporating respectively NotI and AscI restrictions sites. The 413 bp PCR product was TA-cloned into pGEM®-TEasy vector following the recommendation to users protocol (Invitrogen). The 392 bp GBSS cDNA fragment (FIG. 7) was excised from pGEM®-TEasy vector by NotI-AscI digestion then the GBSS insert ligated into a NotI-AscI digested and dephosphorylated pgR106 vector to generate the construct PVX.GBSSas.

As previously described for pds and zep silencing, 6 potato (Solanum tuberosum cv Desiree) plantlets were agro-infiltrated with each of PVX.ZEPas and PVX.GFP constructs. Plants were left to grow and develop tubers in glasshouse conditions as described in the Experimental Procedures section. By 3 months post infection, mature tubers were harvested from each plant. In parallel, GBSS silencing was performed on in vitro propagated potato cv Desiree plantlets as previously described for pds silencing. In vitro generated microtubers derived from in vitro control (PVX.GFP infected) and GBSS silenced (PVX.GBSSas) plants were collected after 9-weeks of culture.

Lugol staining was performed as previously described (Kuipers et al., 1994). At least two tubers or ten microtubers originating from at least two independent control or GBSS silenced plants were analysed. A two- to three-mm tuber slice was dropped into a freshly made Lugol solution (1% Lugol's solution, I-KI [1:2, v/v}, Merck) and remained in contact for 10 minutes. The stained tuber slice surface was scraped with a sterile scalpel blade to isolate tuber tissue that was then resuspended in a drop of distilled water and mounted into microscope slides prior to microscopy. Slides were observed under light microscope Nikon Optiphot I (Nikon, Kingston, UK) and pictures were taken using a Colour Video Camera KY-F55B (Photonic Science, Robertsbridge, UK) using software ImageProPlus 4.1 (Media Cybernetics, Silver Spring, Md., USA).

Pale-red concentric growth rings with blue core (FIG. 8 upper right panel) and mixed thin-blue and red concentric growth rings with blue core (FIG. 8 lower right panel) from iodine-stained starch granules originating from PVX.GBSSas (FIG. 8 upper and lower right panels) infected plants were observed. This staining pattern is characteristic of reduced amylose content as a consequence of GBSS silencing in microtubers and tubers starch granules. In contrast, iodine staining pattern of tubers starch granules appears blue after Lugol staining, indicative of an unaltered amylose content in unsilenced control microtubers and tubers (FIG. 8 PVX.GFP upper and lower left panels respectively).

In this study, the effect of inhibition of GBSS gene expression by PVX VIGS approach was observed in starch granules of both tubers and in vitro generated microtubers.

The results illustrate the flexibility and the robustness of the PVX VIGS based approach where GBSS silencing phenotype was comparable to those obtained with transgenic plants expressing GBSS in antisense orientation. As previously reported, tuber starch granules with blue-staining cores of varying size were also observed after the antisense inhibition of GBSS in transgenic plants (Kuipers et al., 1994). It was proposed that this distinctive pattern could result from the decrease of GBSS protein content between the core and the surface of the granule in combination with the appositional mode of growth of starch granules. Due to an increase in total granule surface during development, the amount of GBSS protein might be sufficient for the normal level of amylase production until a certain size is reached. From this point, which would depend on the level of inhibition of GBSS gene expression, the available GBSS protein may become limiting for adequate synthesis of amylose throughout the outer growth rings of the granule (Kuipers et al., 1994).

Taken together, the examples of successful VIGS of endogenous genes associated with carotenoid and amylose biosynthetic pathways described above illustrate the robustness of the PVX VIGS approach for rapid reverse and forward genetics in order to study and manipulate tuber-associated traits in both glasshouse-grown tubers and in vitro generated microtubers. This approach will be suitable to manipulate other biosynthetic pathways, and to study the effects of developmental, biotic and abiotic stimuli on tuber-associated traits for example.

This study represents the first demonstration of VIGS-mediated down-regulation of gene expression in both diploid and tetraploid Solanum species. Here we report that the carotenoid biosynthetic pathway, where PDS is one of the early steps, can be manipulated by extending the use of a previously described PVX VIGS vector to a different plant host tolerating sufficient levels of PVX accumulation. In parallel, a previously described tobacco rattle virus (TRV, Ratcliff et al, 2001) vector was also tested. However, it did not reach detectable accumulation levels nor did it induce VIGS in the selected tetraploid S. tuberosum cultivars (data not shown). In contrast, recombinant PVX.PDS_{AS} vector was detected by RT-PCR in silenced microtubers and in all others silenced organs, including leaves and tubers (data not shown). This emphasises the prerequisite of a threshold of virus and or dsRNA accumulation to generate an effective VIGS response.

The microtuberization system, in conjunction with VIGS, has a number of potential benefits compared with analysis of tubers produced conventionally in glasshouse conditions. Microtubers develop rapidly in a relatively synchronous manner under controlled tissue-culture conditions. This enables easier identification of tuber phenotype alterations, making it more amenable for characterization of gene function.

The effectiveness of VIGS in potato tubers enables high throughput analysis of gene function to identify genes involved in important traits such as tuber development, metabolism and pathogen resistance. Furthermore, this reverse genetic VIGS-approach will be particularly powerful in combination with analyses of the transcriptome and metabolome.

### REFERENCES

Aoyama, T and Chua, N H (1997) A glucocorticoid-mediated transcriptional induction system in transgenic plants. The Plant Journal 11, 605-612.
Baulcombe, D C (1999) Fast forward genetics based on virus-induced gene silencing. Curr Opin Plant Biol 2: 109-113.
Birch, P R J, Avrova, A O, Lyon, G D, Duncan, J M, Toth, R L (1999) Isolation of potato genes that are induced during an early stage of the hypersensitive response to Phytophthora infestans. Mol Plant-Microbe Interact 12: 356-361.
Brunt, A A, Crabtree, K, Dallwitz, M J, Gibbs, A J, Watson, L, Zarcher, E J (1996) Plant Viruses Online: Descriptions and Lists from the VIDE Database. In Brunt, A A, Crabtree, K, Dallwitz, M J, Gibbs, A J, Watson, L and Zurcher, E J, eds, URL http://biology.anu.edu.au/Groups/MES/vide/.
Busch, M, Seuter, A, Hain, R (2002) Functional analysis of the early steps of carotenoid biosynthesis in tobacco. Plant Physiol. 128, 439-453.
Chapman, S N, Kavanagh, T A and Baulcombe, D C (1992) Potato virus X as a vector for gene expression in plants. The Plant Journal 2, 549-557.
Crameri, A, Whitehorn, E A, Tate, E, Stemmer, W P C (1996) Improved green fluorescent protein by molecular evolution using DNA shuffling. Nature Biotechnol 14: 315-319.
Fraser, P D, Pinto, M E, Holloway, D E, Bramley, P M (2000) Technical advance: application of high-performance liquid chromatography with photodiode array detection to the metabolic profiling of plant isoprenoids. Plant J 24: 551-558.
Goldbach, R, Bucher, E, Prins, M (2003) Resistance mechanisms to plant viruses: an overview. Virus Res 92: 207-212.
Hellens, R P, Edwards A E, Leyland, N R, Bean, S, Mullineaux, P M (2000) pGreen: a versatile and flexible binary Ti vector for Agrobacterium-mediated plant transformation. Plant Mol Biol 42: 819-832.
Holzberg, S, Brosio, P, Gross, C, Pogue, G P (2002) Barley stripe mosaic virus-induced gene silencing in a monocot plant. Plant J 30: 315-327.
Jones, L, Hamilton, A J, Voinnet, O, Thomas, CL, Maule, A J, Baulcombe, D C (1999) RNA-DNA interactions and DNA methylation in post-transcriptional gene silencing. Plant Cell 11: 2291-2301.
Kuipers, A G J, Jacobsen, E, Visser, R G F (1994) Formation and deposition of amylase in the potato tuber starch granule are affected by the reduction of granule-bound starch synthase. The Plant Cell 6, 43-52.
Kumagai, M H, Donson, J, della-Cioppa, G, Harvey, D, Hanley, K. Grill, L K (1995) Cytoplasmic inhibition of carotenoid biosynthesis with virus-derived RNA. Proc Natl Acad Sci USA 92: 1679-1683.
Lacomme, C, Santa Cruz, S (1999) Bax-induced cell death in tobacco is similar to the hypersensitive response. Proc Natl Acad Sci USA 96: 7956-7961.
Lacomme, C, Hrubikova, K, Hein, I (2003) Enhancement of virus-induced gene silencing through viral-based production of inverted-repeats. Plant J 34: 543-553.
Liu, Y, Schiff, M, Dinesh-Kumar, SP (2002) Virus-induced gene silencing in tomato. Plant J 31: 777-786.
Lu, R, Martin-Hernandez, A M, Peart, J R, Malcuit, I, Baulcombe, D C (2003a) Virus-induced gene silencing in plants. Methods 30: 296-303.
Lu, R, Malcuit, I, Moffett, P, Ruiz, M T, Peart, J, Wu, A J, Rathjen, J P, Bendahmane, A, Day, L, Baulcombe, D C (2003b) High throughput virus-induced gene silencing implicates heat shock protein 90 in plant disease resistance. EMBO J 22: 5690-5699.
Morris, W L, Ducreux, L, Griffiths, D W, Davies, H V, Taylor, M A (2004) Carotenogenesis during tuber development and storage in potato. J Exp Bot 55: 975-982.
Muller-Rober, B, Sonnewald, U, Willmitzer, L (1992) Inhibition of the ADP-glucose pyrophosphorylase in transgenic potatoes leads to sugar-storing tubers and influences tuber formation and expression of tuber storage protein genes. EMBO J 11, 1229-1238.
Murashige, T, Skoog, F (1962) A revised medium for rapid growth and bioassays with tobacco tissue cultures. Plant Physiol 15: 473-497.
Odell, J T, Nagy, F, Chua, N H (1985) Identification of DNA sequences required for activity of the cauliflower mosaic virus 35S promoter. Nature, 313, 810-812.
Ratcliff, F, Martin-Hernandez, A M. Baulcombe, D C (2001). Tobacco rattle virus as a vector for analysis gene function by silencing. Plant J 25: 237-245.
Romer, S, Lubeck, J, Kauder, F, Steiger, S, Adomat, C, Sandmann, G (2002) Genetic engineering of a zeaxanthin-rich potato by antisense inactivation and co-suppression of carotenoid epoxidation. Metabolic Engineering 4: 263-272
Roslan, H A, Salter, M G, Wood, C D, Caddick, M X et al (2001) Characterization of the ethanol-inducible alc gene-expression system in Arabidopsis thaliana. The Plant Journal 28, 225-235.
Santa Cruz, S, Chapman, S, Roberts, A G, Roberts, I M, Prior, D A M, Oparka, K J (1996) Assembly and movement of a plant virus carrying a green fluorescent protein overcoat. Proc Natl Acad Sci USA 93: 6286-6290.
Song, J, Bradeen, J M, Jiang, J et al (2003) Gene RB cloned from Solanum bulbocastanum confers broad spectrum resistance to potato late blight. Proc Natl Acad Sci USA 100: 9128-9133.
Takken, F L W, Luderer, R, Gabriels, S H E J, Westerink, N, Lu, R, de Wit, P J G M, Joosten, M H A J (2000) A functional cloning strategy, based on a binary PVX-expression vector, to isolate HR-inducing cDNAs of plant pathogens Plant J 24: 275-283.
Voinnet, O, Pinto, Y M, Baulcombe D C (1999) Suppression of gene silencing: a general strategy used by diverse DNA and RNA viruses. Proc Natl Acad Sci USA 96: 14147-14152.
Voinnet, O (2001) RNA silencing as a plant immune system against viruses. Trends in Genet 17: 449-458.
Xu, X, van Lammeren, A A M, Vermeer, E, Vreugdenhil, D (1998) The role of gibberellin, abscissic acid, and sucrose in the regulation of potato tuber formation in vitro. Plant Physiol 117: 575-584.

1. A recombinant vector comprising the vector pGR106 and including a polynucleotide silencing sequence complementary or homologous to at least part of a target gene.

2. The vector as claimed in claim 1 wherein the silencing sequence is complementary or homologous to at least 60% of the full target gene mRNA transcript sequence.

3. The vector as claimed in claim 2 wherein the silencing sequence is complementary or homologous to at least 80% of the full target gene mRNA transcript sequence.

4. The vector as claimed in claim 3 wherein the silencing sequence is complementary or homologous to at least 95% of the full target gene mRNA transcript sequence.

5. The vector as claimed in claim 1 wherein said silencing sequence is complementary or homologous to a target gene which is an endogenous gene of a host cell.

6. The vector as claimed in claim 1 wherein said silencing sequence is complementary or homologous to a target gene which is expressed in a plant host cell.

7. The vector as claimed in claim 6 wherein said target gene is expressed in tuber cells.

8. The vector as claimed in claim 1 wherein said silencing sequence is complementary or homologous to a target gene which is expressed in Solanum spp.

9. The vector as claimed in claim 1 wherein said silencing sequence is complementary and homologous to a gene involved in the carotenoid biosynthetic pathway.

10. The vector as claimed in claim 9 wherein said gene is the zeaxanthin epoxidase gene.

11. The vector as claimed in claim 10 wherein said silencing sequence is SEQ ID No. 13.

12. The vector as claimed in claim 1 wherein the silencing sequence is complementary or homologous to a gene involved in starch biosynthesis.

13. The vector as claimed in claim 12 wherein said gene is the granule-bound starch synthase gene.

14. The vector as claimed in claim 13 wherein said silencing sequence is SEQ ID No. 14.

15. A method of silencing a target gene expressed by Solanum spp., said method comprising operably linking a polynucleotide silencing sequence which is complementary or homologous to at least part of said target gene into a vector, wherein said vector is derived from pGR106.

16. The method as claimed in claim 15, wherein the silencing sequence is complementary or homologous to at least 60% of the full target gene mRNA transcript sequence.

17. The method as claimed in claim 16, wherein the silencing sequence is complementary or homologous to at least 80% of the full target gene mRNA transcript sequence.

18. The method as claimed in claim 17, wherein the silencing sequence is complementary or homologous to at least 95% of the full target gene mRNA transcript sequence.

19. The method, as claimed in claim 15 wherein the target gene is an endogenous gene of Solanum spp.

20. The method as claimed in claim 15 wherein the target gene is expressed in a tuber or microtuber of Solanum spp.

21. The method as claimed claim 15 wherein the target gene involved in the carotenoid biosynthetic pathway or starch biosynthesis.

22. The method as claimed in claim 21 wherein the target gene is the zeaxanthin epoxidase gene.

23. The method as claimed in claim 22 wherein the silencing sequence is SEQ ID No. 13.

24. The method as claimed in claim 21 wherein the target gene is the granule-bound starch synthase gene.

25. The method as claimed in claim 24 wherein the silencing sequence is SEQ ID No. 14.

26. A method of producing a VIGS vector able to induce gene silencing of a target gene in a plant host cell, said method comprising operably linking a polynucleotide silencing sequence which is complementary or homologous to said target gene to a promoter in the vector pGR106.

27. The method as claimed in claim 26 wherein the silencing sequence is complementary or homologous to at least 60% of the full target gene mRNA transcript sequence.

28. The method as claimed in claim 27 wherein the silencing sequence is complementary or homologous to at least 80% of the full target gene mRNA transcript sequence.

29. The method as claimed in claim 28 wherein the silencing sequence is complementary or homologous to at least 95% of the full target gene mRNA transcript sequence.

30. The method as claimed in claim 26 wherein said plant host cell forms part of a whole plant or microplant.

31. The method as claimed in claim 26 wherein said plant host cell is a cell of Solanum spp.

32. The method as claimed in claim 31 wherein said target gene is expressed in a tuber or microtuber.

33. The method as claimed in claim 26 wherein the target gene is involved in the carotenoid biosynthetic pathway or starch biosythesis biosynthesis.

34. The method as claimed in claim 33 wherein, the target gene is the zeaxanthin epoxidase gene.

35. The method as claimed in claim 34 wherein the silencing sequence is SEQ ID No. 13.

36. The method as claimed in claim 33 wherein the target gene is the granule-bound starch synthase gene.

37. The method as claimed in claim 36 wherein the silencing sequence is SEQ ID No. 14.

38. A host plant cell transfected with a recombinant vector as claimed in claim 1.

39. The host plant cell as claimed in claim 38 which is a cell of Solanum spp.

40. A whole plant comprising a host plant cell as claimed in either one of claims 38 and 39.

41. A microplant comprising a host plant cell as claimed in either one or claims 38 and 39.


SEQUENCE LISTING (14 sequences)

SEQ ID NO: 1 - DNA, 20 bp, Artificial
  Primer RTPoPDSfor
ctcgaggtcgtcttctttgg

SEQ ID NO: 2 - DNA, 20 bp, Artificial
  Primer RTPoPDSrev
gtttagttgggcgtggagaa

SEQ ID NO: 3 - DNA, 17 bp, Artificial
  Primer RTPoUBIfor
gcagttggaggacggac

SEQ ID NO: 4 - DNA, 20 bp, Artificial
  Primer RTPoUBIrev
ggccatcttccaactgtttc

SEQ ID NO: 5 - DNA, 26 bp, Artificial
  Primer StPDSfwd
ccaagaccagagctagacaatacagt

SEQ ID NO: 6 - DNA, 15 bp, Artificial
  Primer StPDSrev
ccacccaaacctgca

SEQ ID NO: 7 - DNA, 26 bp, Artificial
  Primer StUBIfwd
acaccattgataatgtcaaggctaag

SEQ ID NO: 8 - DNA, 20 bp, Artificial
  Primer StUBIrev
gccatcctccaattgctttc

SEQ ID NO: 9 - DNA, 22 bp, Artificial
  Primer
tgtactaaagaaatccccatcc

SEQ ID NO: 10 - DNA, 22 bp, Artificial
  Primer
tattgtatcctccccaacagca

SEQ ID NO: 11 - DNA, 25 bp, Artificial
  Primer
aaaagcggccgcagaaatgggagac

SEQ ID NO: 12 - DNA, 26 bp, Artificial
  Primer
aaaaggcgcgcccgcatatatattag

SEQ ID NO: 13 - DNA, 428 bp, Solanum phureja
  misc_feature (407)..(407): n is a, c, g, or t
gattaatgcattggctgctttagaagcaattgatatggatgttgctgaagacatcatgaatgctggctgcatcactggtcaaaggattaatggcttggttgatggtatttctggcaactggtattgcaagtttgatacgttcactccagcagtggaacgtggacttcccgtgacaagagtcatcagccgcatgactttgcagcagatccttgcacgtgctgttggggaggatacaattatgaatgaaagtaatgttgtagactttgaggatgatggggagaaggttagtgtggttcttgagaatggacaacgatttacaggtgatcttctggttggtgctgatggcataaggtctaaggtacggactaatttgtttggacccagtgaagttacttactctggctacncttgttatactggaattaatc

SEQ ID NO: 14 - DNA, 392 bp, Solanum tuberosum
agaaatgggagacattgctattgggcttaggagcttctggcagtgaacccggtgttgaaggggaagaaatcgctccacttgccaaggaaaatgtagccactccctaaatgagctttggttatccttgtttcaacaataagatcattaagcaaacgtatttactagcgaactatgtagaaccctattatggggtctcaatcatctacaaaatgattggtttttgctggggagcagcagcatattaggctgtaaaatcctggttaatgattttgtaggtaagggctatttaaggttgtgtggatcaaagtcaatagaaaatagttattactaacgtttgcaactaaatacttagtaatgtagcataaataatactagtagctaatatatatgcg